(19) United States
(12) Patent Application Publication
(10) Pub. No.: US 2021/0038670 A1
(43) Pub. Date: Feb. 11, 2021

(54) COMPOSITION AND METHODS

(71) Applicant: Jacob Austin Vandersteen, Maple Grove, MN (US)

(72) Inventor: Jacob Austin Vandersteen, Maple Grove, MN (US)

(21) Appl. No.: 16/984,329

(22) Filed: Aug. 4, 2020

Related U.S. Application Data
(60) Provisional application No. 62/883,287, filed on Aug. 6, 2019.

(51) Int. Cl. A61K 36/42; A61K 31/198; A61K 31/05; A61K 31/12; A61K 31/19; A61K 31/4172; A61K 31/405; A61K 33/06; A61K 33/30; A61K 33/00; A61K 9/00

(52) U.S. Cl. CPC A61K 36/42; A61K 31/198; A61K 31/05; A61K 31/12; A61K 31/19; A61K 9/0095; A61K 31/405; A61K 33/06; A61K 33/30; A61K 33/00; A61K 31/4172

(57) ABSTRACT

The present disclosure provides a composition, e.g., a powder or liquid beverage, including L-arginine, resveratrol, curcumin, and luo han guo extract containing mogrosides. Also provided is a method of making the composition, including mixing components into a composition comprising water at certain temperatures and forming a solution. A method of improving circulation is further provided, including administering the composition to a subject, such as a human or an animal.

### TECHNICAL FIELD

The present disclosure relates to compositions (e.g., beverages) and methods of making and using same.

### BACKGROUND

Compositions, such as beverages, have been used for purposes including, for example, one or more of hydration, nutrition, energy, and enjoyment. In some compositions, at least one ingredient may be used to provide a health benefit to the consumer. However, certain compositions include visible particulates that settle during storage and require shaking prior to consumption to disperse the particulates more evenly throughout the composition.

There remains a need for further developments in compositions and their preparation.

### SUMMARY

The present disclosure provides compositions, methods of making compositions, and methods of improving circulation in a subject.

In a first aspect, a composition is provided. The composition includes a) L-arginine; b) resveratrol; c) curcumin; and d) luo han guo extract containing mogrosides. In some embodiments, the composition further includes water and each of a) through d) is dissolved in the water. In some embodiments, the composition is in the form of a powder and each of a) through d) is blended together.

In a second aspect, a method of making a composition is provided. The method includes a) heating a first composition comprising water having a temperature of 165 to 180 degrees Fahrenheit; b) mixing curcumin with the first composition having a temperature of 165 to 180 degrees Fahrenheit to form a second composition; c) mixing luo han guo extract comprising mogrosides into the second composition having a temperature of 145 to 150 degrees Fahrenheit to form a third composition; d) mixing resveratrol and L-arginine into the third composition having a temperature of 120 to 130 degrees Fahrenheit to form a fourth composition; and d) mixing the fourth composition to form a solution.

In a third aspect, a method of improving circulation in a subject is provided. The method includes administering to the subject a composition including 1) L-arginine; 2) resveratrol; 3) curcumin; and 4) luo han guo extract containing mogrosides.

Various unexpected results and advantages are obtained in exemplary embodiments of the disclosure. One such advantage of exemplary embodiments of the present disclosure is provision of a consumable composition that combines nutritional content, positive effect(s) on circulation, and no more than a few calories per serving. Another advantage of exemplary embodiments of the present disclosure is the ability to prepare a composition having low turbidity (e.g., high clarity) that contains components that are traditionally difficult to dissolve or keep suspended in an aqueous composition.

The above summary of the present disclosure is not intended to describe each disclosed embodiment or every implementation of the present disclosure. The description that follows more particularly exemplifies illustrative embodiments. In several places throughout the application, guidance is provided through lists of examples, which examples can be used in various combinations. In each instance, the cited list serves only as a representative group and should not be interpreted as an exclusive list.

### BRIEF DESCRIPTION OF THE DRAWINGS

FIG. 1 is a flow chart of a method of making a composition according to an exemplary embodiment.

While the above-identified figure sets forth an embodiment of the disclosure, other embodiments are also contemplated, as noted in the description. In all cases, this disclosure presents the invention by way of representation and not limitation. It should be understood that numerous other modifications and embodiments can be devised by those skilled in the art, which fall within the scope and spirit of the principles of the present disclosure.

### DETAILED DESCRIPTION

For the following Glossary of defined terms, these definitions shall be applied for the entire application, unless a different definition is provided in the claims or elsewhere in the specification.

### Glossary

Certain terms are used throughout the description and the claims that, while for the most part are well known, may require some explanation. It should be understood that, as used herein:

As used herein, “liquid” refers to the phase of matter that is intermediate between solid and gaseous, and encompasses fluids including solutions, dispersions, and emulsions.

As used herein, “dissolved” refers to a component being soluble in a composition such that the resulting composition has a turbidity of 20 nephelometric turbidity units (NTU) or less, 18 NTU or less, 16 NTU or less, 14 NTU or less, 12 NTU or less, 10 NTU or less, 9 NTU or less, 8 NTU or less, 7 NTU or less, 6 NTU or less, 5 NTU or less, or even 4 NTU or less, as determined using the Turbidity Test in the examples below.

As used herein, “powder” refers to bulk fine solid granular material. Often, a powder is formed by comminution (e.g., by pulverizing, crushing, grinding, etc.) of a larger solid material, such as a plurality of coarse particles.

As used in this specification and the appended embodiments, the singular forms “a”, “an”, and “the” include plural referents unless the content clearly dictates otherwise. Thus, for example, reference to “a material” includes a mixture of two or more materials.

As used in this specification and the appended embodiments, the term “or” is generally employed in its sense including “and/or” unless the content clearly dictates otherwise. The term “and/or” means either or both. For example, the expression “A and/or B” means A, B, or a combination of A and B.

Unless otherwise indicated, all numbers expressing quantities, measurement of properties, and so forth used in the specification and embodiments are to be understood as being modified in all instances by the term “about.” Accordingly, unless indicated to the contrary, the numerical parameters set forth in the foregoing specification and attached listing of embodiments can vary depending upon the desired properties sought to be obtained by those skilled in the art utilizing the teachings of the present disclosure. At the very least, and not as an attempt to limit the application of the doctrine of equivalents to the scope of the claimed embodiments, each numerical parameter should at least be construed in light of the number of reported significant digits and by applying ordinary rounding techniques.

The term “comprises” and variations thereof do not have a limiting meaning where these terms appear in the description and claims.

The words “preferred” and “preferably” refer to embodiments of the disclosure that may afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the disclosure.

Reference throughout this specification to “one embodiment,” “certain embodiments,” “one or more embodiments” or “an embodiment,” whether or not including the term “exemplary” preceding the term “embodiment,” means that a particular feature, structure, material, or characteristic described in connection with the embodiment is included in at least one embodiment of the certain exemplary embodiments of the present disclosure. Thus, the appearances of the phrases such as “in one or more embodiments,” “in certain embodiments,” “in one embodiment,” “in many embodiments” or “in an embodiment” in various places throughout this specification are not necessarily referring to the same embodiment of the certain exemplary embodiments of the present disclosure. Furthermore, the particular features, materials, or characteristics may be combined in any suitable manner in one or more embodiments.

Various exemplary embodiments of the disclosure will now be described. Exemplary embodiments of the present disclosure may take on various modifications and alterations without departing from the spirit and scope of the disclosure. Accordingly, it is to be understood that the embodiments of the present disclosure are not to be limited to the following described exemplary embodiments, but are to be controlled by the limitations set forth in the claims and any equivalents thereof.

In a first aspect, a composition is provided. The composition comprises:

a) L-arginine;

b) resveratrol;

c) curcumin; and

d) luo han guo extract comprising mogrosides.

In some embodiments, the composition further comprises water, and each component a) through d) is dissolved in the water. In some embodiments, each component a) through d) is blended together and the composition is in the form of a powder.

In embodiments in which the composition is in the form of a (solid) powder, the powdered composition is added to a user's desired liquid (e.g., water) and dissolves substantially or completely fully in the liquid, prior to consuming the composition in a liquid form. Typically, moderate shaking or mixing provides an emulsion that remains suspended without forming visible aggregations or coalescence for at least 10 minutes upon standing. This is in part due to the blending process that occurs during the production of the composition. More particularly, the powder composition is passed through various stages of grinding and blending, including using bladed instruments that decrease the particle sizes of the various components and increase separation of the components, as well as tumbling the powder to maximize homogeneity throughout the powder composition. In an embodiment, each individual component is first ground in a grinder to break up aggregates and clumps, next all the components are blended together in a blender, and then the blended components are tumbled in a container.

Components of the composition are described in detail below.

In most embodiments in which the composition comprises water, the water is alkaline water. Alkaline water is water that has been ionized, either through electrolysis or by contacting minerals (e.g., spring water than passes over rocks). A benefit of water having a basic pH is to assist in maintaining the base form of the amino acids. Often, the composition has a pH of 7.1 or greater, 7.2 or greater, 7.3 or greater, 7.4 or greater, 7.5 or greater, 7.6 or greater, 7.7 or greater, 7.8 or greater, 7.9 or greater, or 8 or greater. The pH of the composition is typically 10 or less, 9.8 or less, 9.6 or less, 9.4 or less, 9.2 or less, 9 or less, 8.8 or less, 8.6 or less, or 8.5 or less.

Amino acids can be absorbed directly into the bloodstream to increase available energy more rapidly than proteins that must be digested to break the protein down into amino acids. Amino acids facilitate nearly every process in the human body, for instance being involved in restoring and healing body tissues. Specific amino acids, such as tryptophan, can also act as precursors to neurotransmitters. The amino acids are preferably provided in their base form, which has been documented to be 6.66 times more effective than when consumed in their standard form (see, e.g., Hsamine Kobayashi's BIO Clinica, 29 (1): 63-67, 2014.). Amino acids have been shown to provide advantages such as promoting muscle development, improving bone strength, increasing fat burning, protecting immune function, and improving cardiovascular health. Essential amino acids together comprise the structure of all proteins. Moreover, adding essential amino acids to the diet of an “at-risk” population, such as the elderly, has been shown to demonstrably reduce occurrences of osteoporosis and sarcopenia.

L-arginine is an essential amino acid that stimulates nitric oxide production in the blood. When consumed, L-arginine also influences positive human growth hormone levels and increases natural blood flow, especially when taken prior to exercise. L-arginine is commonly found in high concentrations in red meat. L-arginine stimulates vasodilation (i.e., the widening of blood vessels), allowing blood to flow more readily and decreasing blood pressure. Additionally, it is known that a vegetable based diet is a poor source of L-arginine, thus its inclusion can be particularly beneficial for subjects following a vegan diet. L-arginine is typically present in the composition in an amount of 50 milligrams (mg) or greater per serving, 75 mg or greater per serving, 100 mg or greater per serving, 125 mg or greater per serving, 150 mg or greater per serving, 175 mg or greater per serving, 200 mg or greater per serving, 250 mg or greater per serving, 300 mg or greater per serving, 350 mg or greater per serving, 400 mg or greater per serving, 450 mg or greater per serving, 500 mg or greater per serving, 550 mg or greater per serving, 600 mg or greater per serving, or 650 mg or greater per serving; and 4 grams (g) or less per serving, 3.5 g or less per serving, 3 g or less per serving, 2.5 g or less per serving, 2 g or less per serving, 1.5 g or less per serving, 1 g or less per serving, 800 mg or less per serving, or 600 mg or less per serving.

In addition to the amino acid L-arginine, in some embodiments, the composition further comprises at least one amino acid selected from the group consisting of L-leucine, L-lysine, L-valine, L-isoleucine, L-threonine, L-phenylalanine, L-methionine, L-histidine, and L-tryptophan. In select embodiments, the composition comprises each of L-leucine, L-lysine, L-valine, L-isoleucine, L-threonine, L-phenylalanine, L-methionine, L-histidine, and L-tryptophan (e.g., together, essential amino acids). This selection of amino acids advantageously provides a complete profile.

Preferably, each amino acid is present in an amount of 5 mg or greater per serving, 10 mg or greater, 20 mg or greater, 50 mg or greater, or 100 mg or greater per serving. In some embodiments, the L-leucine is present in an amount of 50 mg to 4 g per serving. In some embodiments, the L-lysine is present in an amount of 50 mg to 4 g per serving. In some embodiments, the L-isoleucine is present in an amount of 50 mg to 4 g per serving. In some embodiments, the L-threonine is present in an amount of 20 mg to 4 g per serving. In some embodiments, the L-phenylalanine is present in an amount of 20 mg to 4 g per serving. In some embodiments, the L-methionine is present in an amount of 5 mg to 4 g per serving. In some embodiments, the L-histidine is present in an amount of 10 mg to 4 g per serving. In some embodiments, the L-tryptophan is present in an amount of 10 mg to 4 g per serving. In some embodiments, the amino acids are present in a ratio of 800 to 10 parts by weight of L-leucine: 800 to 10 parts by weight of L-lysine: 800 to 10 parts by weight of L-isoleucine: 800 to 4 parts by weight L-threonine: 800 to 4 parts by weight of L-phenylalanine: 800 to 1 parts by weight of L-methionine: 800 to 2 parts by weight L-histidine: 800 to 2 parts by weight L-tryptophan, based on the total parts of amino acids. In some embodiments, the amino acids are present in a ratio of 100 to 10 parts by weight of L-leucine: 100 to 10 parts by weight of L-lysine: 100 to 10 parts by weight of L-isoleucine: 100 to 4 parts by weight L-threonine: 100 to 4 parts by weight of L-phenylalanine: 100 to 1 parts by weight of L-methionine: 100 to 2 parts by weight L-histidine: 100 to 2 parts by weight L-tryptophan, based on the total parts of amino acids. In some embodiments, the amino acids are present in a ratio of 20 to 10 parts by weight of L-leucine: 20 to 10 parts by weight of L-lysine: 20 to 10 parts by weight of L-isoleucine: 20 to 4 parts by weight L-threonine: 20 to 4 parts by weight of L-phenylalanine: 20 to 1 parts by weight of L-methionine: 20 to 2 parts by weight L-histidine: 20 to 2 parts by weight L-tryptophan, based on the total parts of amino acids. In a select embodiment, the amino acids are present in a ratio of 51 parts by weight of L-leucine: 22 parts by weight of L-lysine: 22 parts by weight of L-isoleucine: 12 parts by weight L-threonine: 7.5 parts by weight of L-phenylalanine: 3.5 parts by weight of L-methionine: 2.2 parts by weight L-histidine: 1 part by weight L-tryptophan, based on the total parts of amino acids.

Additional optional amino acids include for instance and without limitation, aspartic acid, glycine, D-arginine, glutamic acid, proline, theanine, cysteine, cystine, alanine, tyrosine, arabinose, trans-4-hydroxyproline, asparagine, serine, ornithine, carnitine, glutamine, hydroxyproline, taurine, norvaline, and sarcosine. In some embodiments, the composition comprises at least one of theanine, glycine, or arginine (e.g., theanine) in an amount of 12 mg or greater per serving, 15 mg or greater per serving, 17 mg or greater per serving, 20 mg or greater per serving, 22 mg or greater per serving, or 25 mg or greater per serving; and 100 mg or less per serving. Optionally, the composition comprises each of theanine, glycine, and arginine. Theanine, for instance, is a non-essential amino acid that is noted to naturally improve focus and has been documented to put the brain in an “alpha-wave state” that enhances creativity, and calm attention. Theanine is typically found in green tea. Further, glycine is known to beneficially remove ammonia from the body.

The amino acids may be natural or synthetic. Suitable amino acids can be extracted from corn, for instance.

Resveratrol has been shown to have an ability to cleanse the body of (e.g., excess) fats. For instance, resveratrol is noted as the primary contributing factor to the “French Paradox”, which allows the French population to subsist on high fat diets, while also having a low occurrence of heart disease. Resveratrol is found in red wine and may have effectiveness in doses of even 2 mg when ingested consistently, and up to about 500 mg per serving. Resveratrol can thus provide a significant benefit at a low concentration. The benefits of resveratrol may include longevity support, improved energy and athletic performance, anti-inflammatory properties, support against diabetes and increased insulin sensitivity, neuroprotective benefits, improved heart and blood circulation and even cancer inhibiting properties. The number of studies documenting the effects of resveratrol has grown over the past few years and suggest that resveratrol may support many processes and functions in the human body. Resveratrol has been shown to mimic the positive effects of caloric restriction, including prevention of diet-induced obesity and an increase in mitochondrial function, physical stamina, and glucose tolerance in mice. Resveratrol is thought to accomplish this through the activation of energy-sensing metabolic regulators 5′ monophosphate-activated protein kinase (AMPK), sirtuin 1 (SIRT1), and peroxisome proliferator-activated receptor gamma coactivator-1 (PCG-1). In contrast, inactivity of these regulators has been demonstrated the detrimental effects of accelerated aging, increase retention and storage of adipose tissue, and low energy levels. Resveratrol increases nitric oxide synthesis, which may be particularly beneficial in combination with the stimulation of nitric oxide production by L-arginine. For instance, when blood flows better, the human system is believed to function better because blood carries oxygen and nutrients to all cells of the body to facilitate absorption of what comes into the body and removal of waste. Preferably, a concentrated and highly bioactive form of resveratrol is used, for instance resveratrol commercially available from PureBulk (Roseburg, OR). Resveratrol is typically present in a composition in an amount of 1 mg or greater per serving, 2 mg or greater per serving, 3 mg or greater per serving, 4 mg or greater per serving, 5 mg or greater per serving, 7 mg or greater per serving 10 mg or greater per serving 12 mg or greater per serving, 15 mg or greater per serving, 17 mg or greater per serving, 20 mg or greater per serving, 25 mg or greater per serving, 50 mg or greater per serving, 75 mg or greater per serving, 100 mg or greater per serving, 125 mg or greater per serving, 150 mg or greater per serving, 175 mg or greater per serving, 200 mg or greater per serving, 225 mg or greater per serving, or 250 mg or greater per serving; and 1 g or less per serving, 900 mg or less per serving, 800 mg or less per serving, 700 mg or less per serving, 600 mg or less per serving, 500 mg or less per serving, 400 mg or less per serving, or 300 mg or less per serving.

Mogrosides are derived from luo han guo, a common name for the extract made from the fruit of Siraitia grosvenorii, a herbaceous perennial vine of the Cucurbitaceae family native to China. Luo han guo is also referred to as “monk fruit”, and is primarily known for its sweetness. Indeed, in the form used in the present disclosure, luo han guo is about 300 times sweeter than sugar (e.g., sucrose). Monk Fruit gets its sweetness from mogrosides. Monk Fruit is also noted, however, to provide numerous health benefits of its own. Mogrosides have been documented to inhibit the process of adipogenesis (e.g., the formation of new fat cells). Luo han guo is typically present in a composition in an amount of 50 mg or greater per serving, 60 mg or greater per serving, 70 mg or greater per serving, 80 mg or greater per serving, 90 mg or greater per serving, 99 mg or greater per serving, 100 mg or greater per serving, 110 mg or greater per serving, 120 mg or greater per serving, 130 mg or greater per serving, 150 mg or greater per serving, 175 mg or greater per serving, 200 mg or greater per serving, 225 mg or greater per serving, or 250 mg or greater per serving; and 1,800 mg or less per serving, 1,700 mg or less per serving, 1,600 mg or less per serving, 1,500 mg or less per serving, 1,400 mg or less per serving, 1,300 mg or less per serving, 1,200 mg or less per serving, 1,100 mg or less per serving, 1,000 mg or less per serving, 900 mg or less per serving, 800 mg or less per serving, 700 mg or less per serving, 600 mg or less per serving, 500 mg or less per serving, 400 mg or less per serving, or 300 mg or less per serving.

Curcumin is the active component of turmeric, and similarly to resveratrol, is noted to have many positive health benefits that promote the optimal function of and improve the integrity of the human circulatory system. These two ingredients have been documented to have harmonious chondroprotective benefits that protect and preserve joints and bones as well as to improve and maintain positive mental well-being. In a 2019 study, curcumin was demonstrated to have the ability to increase insulin sensitivity (e.g., facilitate healthy glucose uptake into cells). The curcumin is typically present in a composition an amount of 25 mg or greater per serving, 50 mg or greater per serving, 75 mg or greater per serving, 100 mg or greater per serving, 125 mg or greater per serving, 150 mg or greater per serving, 175 mg or greater per serving, 200 mg or greater per serving, 225 mg or greater per serving, or 250 mg or greater per serving; and 1 g or less per serving, 900 mg or less per serving, 700 mg or less per serving, 500 mg or less per serving, or 300 mg or less per serving.

It is believed that a combination of certain ingredients of the present disclosure can result in a synergistic activation of AMPK. AMPK exists in every cell of the human body and performs many functions such as the inhibition of fat storage, increased insulin sensitivity, and activation of cell metabolism. It has been discovered that in combination with resveratrol and curcumin, mogrosides also activate AMPK. AMPK activation may be determined, for instance, using an assay kit. Suitable assay kits are commercially available, such as under the trade designation “ENZYFLUO” AMPK Phosphorylation Assay Kit from BioAssay Systems (Hayward, Calif.).

Compositions according to at least certain embodiments of the present disclosure further comprise beta-hydroxybutyrate (BHB). BHB is an endogenous ketone body produced in the liver and is thought to be effective in promoting a healthy fat burning state. BHB is utilized in vivo in times of stress, often due to low sugar levels and has been used as a brain and body fuel in the absence of external nutrition. BHB can also be taken as a supplement in addition to amounts produced by the body. BHB is commercially available as a stable salt of calcium, sodium, or magnesium. BHB may be present in a composition an amount of 250 mg or greater per serving, 500 mg or greater per serving, 750 mg or greater per serving, 1 g or greater per serving, 1.25 g or greater per serving, 1.50 g or greater per serving, 1.75 g or greater per serving, 2 g or greater per serving, 2.25 g or greater per serving, or 2.5 g or greater per serving; and 10 g or less per serving, 9 g or less per serving, 7 g or less per serving, 5 g or less per serving, or 3 g or less per serving.

Compositions according to at least certain embodiments of the present disclosure further comprise one or more minerals. Suitable minerals include for instance and without limitation, magnesium, zinc, sodium, potassium, calcium, chlorine, phosphorous, sulfur, chromium, cobalt, copper, fluorine, iron, manganese, molybdenum, selenium, and iodine. In some embodiments, the composition further comprises at least one of magnesium, zinc, sodium, or potassium, for instance each of magnesium, zinc, sodium, and potassium. Each mineral included may individually be present in an amount of 10 to 100 percent of the recommended daily allowance (RDA) of the particular mineral, per serving. Typically, each mineral is included in the form of a salt to improve dissolution of the mineral in a (e.g., aqueous) liquid composition.

Compositions according to at least certain embodiments of the present disclosure further comprise one or more vitamins. Suitable vitamins include for instance and without limitation, beta-carotene (i.e., a precursor to vitamin A), vitamin B3 (i.e., niacin), vitamin B6, vitamin B9, vitamin B12, vitamin C, vitamin D, vitamin D3, and vitamin K. In some embodiments, the composition comprises at least one of beta-carotene, vitamin C, or vitamin D. Optionally, the composition comprises each of beta-carotene, vitamin C, and vitamin D. Further, B-vitamins are preferred vitamins due to their ability to act as co-factors to improved protein synthesis. Each vitamin included may individually be present in an amount of 10 to 100 percent of the RDA of the particular vitamin, per serving.

In some embodiments, the composition further comprises at least one of cannabidiol (CBD) oil, beet root extract, avocado oil extract, ginger root extract, or tetrahydrocannabinol (THC) extract. CBD oil is an oil extracted from cannabis and is known to provide nearly all of the health benefits of marijuana without the psychoactive effects. CBD acts on the natural receptors in the brain to improve the body's ability to relax. Beet root extract is known to increase nitric oxide (NO) content in the body and help decrease blood pressure. Avocado oil contains desirable medium chain triglycerides. Ginger root extract provides a unique taste and is a known antioxidant and anti-inflammatory component. THC extract is THC that has been extracted from cannabis and provides the psychoactive effects of marijuana.

In many embodiments, the composition further comprising caffeine. Caffeine is the most commonly consumed psychoactive ingredient in the world. Doses of up to 200 mg per day have been documented to improve overall focus and clarity. Caffeine tends to prevent adenosine receptors in the brain from receiving a “tired signal”. The caffeine may be present in an amount of 90 mg or greater per serving, which is similar to the amount of caffeine in an 8-ounce cup of coffee. Typically, caffeine is present in an amount of 300 mg or less per serving. Moreover, when ingested in combination with caffeine, L-theanine enhances the positive effects of caffeine, and tends to provide relief for caffeine's potentially negative symptoms such as jitters and racing heartbeat. These two components together have been documented to show an increase in cognitive performance and overall mood.

It is believed that the combination of L-arginine, resveratrol, curcumin, and mogrosides according to embodiments of the present disclosure synergistically works to improve circulation when consumed by a subject (e.g., a human or animal). In some embodiments of the composition, the L-arginine is present in an amount of 50 mg to 4 g, the resveratrol is present in an amount of 1 mg to 1 g, the curcumin is present in an amount of 25 mg to 1 g, and the luo han guo extract is present in an amount of 50 mg to 750 mg. Typically, the L-arginine is present in the composition in an amount of 400 mg or greater per serving, the resveratrol is present in an amount of 2 mg or greater per serving, the curcumin is present in an amount of 100 mg or greater per serving, and the luo han guo extract is present in an amount of 99 mg or greater per serving.

In some embodiments, the L-arginine, curcumin, luo han guo extract, and resveratrol are present in a ratio of 50 to 4,000 parts by weight L-arginine: 25 to 1,000 parts by weight curcumin: 50 to 750 parts by weight luo han guo extract: 1 to 1,000 parts by weight resveratrol, based on the total weight of L-arginine, curcumin, luo han guo extract, and resveratrol. In some embodiments, the L-arginine, curcumin, luo han guo extract, and resveratrol are present in a ratio of 50 to 1,000 parts by weight L-arginine: 25 to 250 parts by weight curcumin: 50 to 188 parts by weight luo han guo extract: 1 to 250 parts by weight resveratrol, based on the total weight of L-arginine, curcumin, luo han guo extract, and resveratrol. In some embodiments, the L-arginine, curcumin, luo han guo extract, and resveratrol are present in a ratio of 50 to 500 parts by weight L-arginine: 25 to 125 parts by weight curcumin: 50 to 94 parts by weight luo han guo extract: 1 to 125 parts by weight resveratrol, based on the total weight of L-arginine, curcumin, luo han guo extract, and resveratrol. In a select embodiment, the L-arginine, curcumin, luo han guo extract, and resveratrol are present in a ratio of 8 parts by weight L-arginine: 2 parts by weight curcumin: 2 parts by weight luo han guo extract: 1 part by weight resveratrol, based on the total weight of L-arginine, curcumin, luo han guo extract, and resveratrol, based on the total weight of L-arginine, curcumin, luo han guo extract, and resveratrol.

In a second aspect, a method of making a composition is provided. The method comprises: comprising:
a) heating a first composition comprising water to a temperature of 165 to 180 degrees Fahrenheit;
b) mixing curcumin with the first composition having a temperature of 165 to 180 degrees Fahrenheit to form a second composition;
c) mixing luo han guo extract comprising mogrosides into the second composition having a temperature of 145 to 150 degrees Fahrenheit to form a third composition;
d) mixing resveratrol and L-arginine into the third composition having a temperature of 120 to 130 degrees Fahrenheit to form a fourth composition; and e) mixing the fourth composition to form a solution.

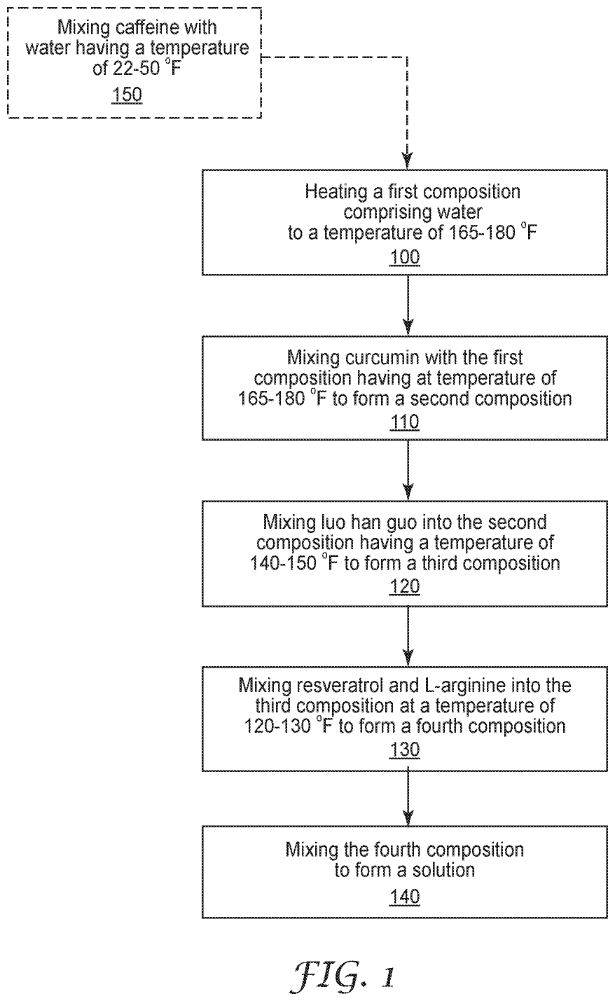

Referring to FIG. 1, a flow chart is provided. FIG. 1 includes Step 100 of heating a first composition comprising water to a temperature of 165-180 degrees Fahrenheit; Step 110 of mixing curcumin with a first composition comprising water having a temperature of 165 to 180 degrees Fahrenheit to form a second composition; Step 120 of mixing luo han guo extract containing mogrosides into the second composition having a temperature of 145 to 150 degrees Fahrenheit to form a third composition; Step 130 of mixing resveratrol and L-arginine into the third composition having a temperature of 120 to 130 degrees Fahrenheit to form a fourth composition; and Step 140 of mixing to form a solution. Typically, when a component is first added within the temperature range noted, the composition is a dispersion, and then following mixing usually the component dissolves and the composition is a solution until the next component is added. Often, the water is alkaline water, as discussed above with respect to the first aspect.

In embodiments in which caffeine is used, optional Step 150 may be performed, of adding caffeine to the first composition (e.g., water) when the first composition is at a temperature of 22 to 50 degrees Fahrenheit. It has been found that heating the first composition, which may consist of water, followed by allowing the composition to cool and adding certain ingredients when the composition has decreased to a particular temperature range, tends to promote effective dissolution of the ingredients and minimize turbidity.

In at least certain embodiments according to the present disclosure, the solution is advantageously clear (e.g., having a turbidity of 20 NTU or less) instead of hazy. In select embodiments, another way of improving clarity involves including at least one lipophilic component in the dispersion, for instance by mixing CBD oil into the dispersion.

In many embodiments, the method forms a concentrated solution that can be diluted to make a ready to drink (RTD) composition. In such instances, the method further comprises diluting the solution with water (e.g., alkaline water) in a ratio of solution: water of 1:10 to 1:20, such as about 1:11, 1:12, 1:13, 1:14, 1:15, 1:16, 1:17, 1:18, 1:19, or 1:20. Following dilution, the composition may be transferred to sterile containers for storage and distribution.

In some embodiments, the resveratrol and L-arginine are ground together prior to mixing into the composition. Similarly, it may be advantageous to blend or grind together whichever ingredients that have a particulate (e.g., solid powder) form that will be added to a composition at about the same temperature or temperature range, for more effective dispersion and dissolution of the ingredients once mixed into the composition.

In a third aspect, a method of improving circulation in a subject is provided. The method comprises:

a) administering to the subject a composition comprising:
1) L-arginine;
2) resveratrol;
3) curcumin; and
4) luo han guo extract comprising mogrosides.

As discussed above, an advantage of embodiments according to the present disclosure is that the combination of L-arginine, resveratrol, curcumin, and mogrosides is believed to synergistically work to improve circulation. In some embodiments, improving circulation includes increasing AMPK activation. In many embodiments, improving circulation includes improving blood flow. Preferably, increasing nitric oxide (NO) stimulation, NO synthesis, or both is achieved in the improved circulation.

In some embodiments, the composition is administered to the subject at least once daily. Typically, the composition is a beverage. For instance, the composition can further comprise water and be provided in RTD form (e.g., with one or two servings contained per bottle). Alternatively, the composition is provided in the form of a powder and a user combines the powder with a liquid of choice (often water) to dissolve and/or suspend the powdered components in the liquid prior to consumption.

Various non-limiting exemplary embodiments according to the present disclosure are provided below.

### Exemplary Embodiments

Embodiment 1 is a composition. The composition includes a) L-arginine; b) resveratrol; c) curcumin; and d) luo han guo extract containing mogrosides.

Embodiment 2 is the composition of embodiment 1, further including water and wherein each of a) through d) is dissolved in the water.

Embodiment 3 is the composition of embodiment 2, wherein the water is alkaline water.

Embodiment 4 is the composition of embodiment 2 or embodiment 3, wherein the composition has a pH of 7.1 or greater.

Embodiment 5 is the composition of embodiment 1, in which each of a) through d) is blended together and the composition is in the form of a powder.

Embodiment 6 is the composition of any of embodiments 1 to 5, further including beta-hydroxybutyrate (BHB).

Embodiment 7 is the composition of any of embodiments 1 to 6, further including at least one amino acid selected from the group consisting of L-leucine, L-lysine, L-valine, L-isoleucine, L-threonine, L-phenylalanine, L-methionine, L-histidine, and L-tryptophan.

Embodiment 8 is the composition of embodiment 7, including each of L-leucine, L-lysine, L-valine, L-isoleucine, L-threonine, L-phenylalanine, L-methionine, L-histidine, and L-tryptophan.

Embodiment 9 is the composition of embodiment 8, wherein the amino acids are present in a ratio of 100 to 10 parts by weight of L-leucine: 100 to 10 parts by weight of L-lysine: 100 to 10 parts by weight of L-isoleucine: 100 to 4 parts by weight L-threonine: 100 to 4 parts by weight of L-phenylalanine: 100 to 1 parts by weight of L-methionine: 100 to 2 parts by weight L-histidine: 100 to 2 parts by weight L-tryptophan, based on the total parts of amino acids.

Embodiment 10 is the composition of embodiment 8 or embodiment 9, wherein each amino acid is present in an amount of 100 mg or greater per serving.

Embodiment 11 is the composition of any of embodiments 1 to 10, further including at least one of theanine, glycine, or arginine.

Embodiment 12 is the composition of embodiment 11, including each of theanine, glycine, and arginine.

Embodiment 13 is the composition of embodiment 11 or embodiment 12, including theanine in an amount of 12 mg or greater per serving.

Embodiment 14 is the composition of any of embodiments 1 to 13, further including at least one of magnesium, zinc, sodium, or potassium.

Embodiment 15 is the composition of embodiment 14, including each of magnesium, zinc, sodium, and potassium.

Embodiment 16 is the composition of any of embodiments 1 to 15, further including at least one of beta-carotene, vitamin C, or vitamin D.

Embodiment 17 is the composition of embodiment 16, including each of beta-carotene, vitamin C, and vitamin D.

Embodiment 18 is the composition of any of embodiments 1 to 17, further including at least one of CBD oil, beet root extract, avocado oil extract, ginger root extract, or THC extract.

Embodiment 19 is the composition of embodiment 18, including CBD oil.

Embodiment 20 is the composition of embodiment 18 or embodiment 19, including THC extract.

Embodiment 21 is the composition of any of embodiments 1 to 20, further including caffeine.

Embodiment 22 is the composition of embodiment 21, wherein the caffeine is present in an amount of 90 mg or greater per serving.

Embodiment 23 is the composition of any of embodiments 1 to 22, wherein the L-arginine is present in an amount of 400 mg or greater per serving, the resveratrol is present in an amount of 2 mg or greater per serving, the curcumin is present in an amount of 100 mg or greater per serving, and the luo han guo extract is present in an amount of 99 mg or greater per serving.

Embodiment 24 is the composition of any of embodiments 1 to 23, wherein the L-arginine, curcumin, luo han guo extract, and resveratrol are present in a ratio of 50 to 1,000 parts by weight L-arginine: 25 to 250 parts by weight curcumin: 50 to 188 parts by weight luo han guo extract: 1 to 250 parts by weight resveratrol, based on the total weight of L-arginine, curcumin, luo han guo extract, and resveratrol.

Embodiment 25 is a method of making a composition. The method includes a) heating a first composition comprising water having a temperature of 165 to 180 degrees Fahrenheit; b) mixing curcumin with the first composition having a temperature of 165 to 180 degrees Fahrenheit to form a second composition; c) mixing luo han guo extract comprising mogrosides into the second composition having a temperature of 145 to 150 degrees Fahrenheit to form a third composition; d) mixing resveratrol and L-arginine into the third composition having a temperature of 120 to 130 degrees Fahrenheit to form a fourth composition; and d) mixing the fourth composition to form a solution.

Embodiment 26 is the method of embodiment 25, further including adding caffeine to the first composition when the first composition is at a temperature of 22 to 50 degrees Fahrenheit.

Embodiment 27 is the method of embodiment 25 or embodiment 26, wherein the water is alkaline water.

Embodiment 28 is the method of any of embodiments 27 to 29, wherein the solution has a turbidity of 5 nephelometric units (NTU) or less, as determined by a turbidity test.

Embodiment 29 is the method of any of embodiments 25 to 28, further including mixing CBD oil into at least one of the first composition, the second composition, the third composition, or the fourth composition.

Embodiment 30 is the method of any of embodiments 25 to 29, further including diluting the solution with water in a ratio of solution: water of 1:10 to 1:20.

Embodiment 31 is the method of any of embodiments 25 to 30, wherein the resveratrol and L-arginine are ground together prior to mixing into the third composition.

Embodiment 32 is a method of improving circulation in a subject. The method includes administering to the subject a composition including 1) L-arginine; 2) resveratrol; 3) curcumin; and 4) luo han guo extract containing mogrosides.

Embodiment 33 is the method of embodiment 32, wherein the composition is administered to the subject at least once daily.

Embodiment 34 is the method of embodiment 32 or embodiment 33, wherein the composition further comprises water and is a beverage.

Embodiment 35 is the method of any of embodiments 32 to 34, wherein the improving circulation includes increasing 5′ adenosine monophosphate-activated protein kinase (AMPK) activation.

Embodiment 36 is the method of any of embodiments 32 to 35, wherein the improving circulation includes improving blood flow.

Embodiment 37 is the method of any of embodiments 32 to 36, wherein the improving circulation includes increasing nitric oxide (NO) stimulation, NO synthesis, or both.

### EXAMPLES

Objects and advantages of this disclosure are further illustrated by the following examples, but the particular materials and amounts thereof recited in these examples, as well as other conditions and details, should not be construed to unduly limit this disclosure.

### Materials

Unless otherwise noted, all parts, percentages, ratios, etc. in the examples and the rest of the specification are by weight. Table 1. (below) lists materials used in the examples and their sources.

TABLE 1
Materials

| Material | Source |
|---|---|
| Alkaline water | Alkaline 88, Scottsdale, AZ |
| Caffeine | PureBulk, Roseburg, OR |
| Curcumin | PureBulk |
| L-arginine | NutriVita, Lake Forest, CA |
| L-theanine | PureBulk |
| Luo han guo | Lakanto, Orem, UT |
| Niacin | PureBulk |
| Resveratrol | PureBulk |
| Flavor | TrueCitrus, Middle River, MD |
| L-isoleucine | NutriVita |
| L-leucine | NutriVita |
| L-lysine | NutriVita |
| L-valine | NutriVita |
| L-threonine | NutriVita |
| L-phenylalanine | NutriVita |
| L-methionine | NutriVita |
| L-tryptophan | NutriVita |
| L-histidine | NutriVita |
| L-tyrosine | NutriVita |
| Magnesium beta- | |
| hydroxybutyrate (Mg BHB) | |

Turbidity Test

A sample was placed in a 40 inch tall Transparent Turbidity Tube with Secchi Disk (Eisco Scientific LLC, Victor, N.Y.). The secchi disk was lowered into the tube until the disk was no longer visible. The location wherein the secchi disk was first no longer visible was read on the side of the turbidity tube as the measured turbidity of the sample.

TABLE 2
Formulations

| | Example 1 | Comparative Example A |
|---|---|---|
| | Amount per 15.9 | Amount per 15.9 |
| | fluid ounces | fluid ounces |
| Ingredient | (milligrams) | (milligrams) |
| Flavor | 800 | 800 |
| Caffeine | 80 | 0 |
| Curcumin | 35 | 35 |
| L-arginine | 50 | 400 |
| L-theanine | 12 | 400 |
| Luo han guo | 997 | 997 |
| Niacin | 5 | 5 |
| Resveratrol | 10 | 10 |
| L-isoleucine | 50 | 400 |
| L-leucine | 50 | 400 |
| L-lysine | 50 | 400 |
| L-valine | 50 | 400 |
| L-threonine | 20 | 400 |
| L-phenylalanine | 20 | 400 |
| L-methionine | 5 | 400 |

### Example 1

A batch vat was filled with alkaline water in an amount of 15.9 fluid ounces of alkaline water per 1 bottle. Each formula ingredient was included in the amount according to Table 2 above, per 15.9 fluid ounces.

Flavors were added directly to an electric grinder (Krups), blended finely and then added to the batch vat (e.g., large pot) at room temp (i.e., 69 degrees Fahrenheit) and mixed with a wooden spoon to form a dispersion. The dispersion was heated on an electric stove and caffeine was added to the heated dispersion at 180° F. The temperature was measured using a thermometer inserted into the dispersion. Following the addition of caffeine, the dispersion was heated to 212° F., then allowed to cool. When the heated dispersion reached 180° F., curcumin and niacin were added to the dispersion and whisked. Next, luo han guo was added to the electric grinder. Resveratrol, L-arginine, and all other ingredients were combined and blended in the electric grinder. The blended ingredients were all added to the dispersion at 135° F. After thorough mixing using a whisk, the solution was cooled naturally to about 60° F. and transferred to an IGLOO 5 Gallon Seat Top Beverage Jug with spigot (Igloo Products Corp, Katy, Tex.). Last, the solution was dispensed into prepared 16 ounce bottles and stored in a refrigerator at about 42° F.

A sample was removed from one bottle and tested for turbidity according to the Turbidity Test. The turbidity of Example 1 was determined to be 4 NTU.

Comparative Example A

Each formula ingredient was included in the amount according to Table 2 above, per 15.9 fluid ounces. One hundred ounces of standard tap water was added to a Vitamix blender model VM0102D (Vitamix, Cleveland, Ohio), (e.g., 3/4 of the blender container volume). The flavor was added to the tap water and blended in. Next, all amino acids, niacin, and luo han guo were measured, combined directly, and blended into a homogeneous mixture. Resveratrol and curcumin were combined directly into the mixing apparatus and blended. The turbidity of a sample of Comparative Example A was measured according to the Turbidity Test and determined to be 500 NTU.

### Example 2

A powdered composition was prepared.

TABLE 3
Formulation for Powdered Composition

| | Example 2 |
|---|---|
| Ingredient | Amount per serving (grams) |
| Flavor | 1 |
| MgBHB | 1 |
| Curcumin | 0.1 |
| L-arginine | 0.2 |
| L-theanine | 0.2 |
| Luo han guo | 0.3 |
| Niacin | 0.02 |
| Resveratrol | 0.05 |
| L-isoleucine | 0.15 |
| L-leucine | 0.15 |
| L-lysine | 0.1 |
| L-valine | 0.15 |
| L-threonine | 0.1 |
| L-phenylalanine | 0.05 |
| L-methionine | 0.02 |
| L-tryptophan | 0.025 |
| L-tyrosine | 0.1 |
| L-histidine | 0.05 |

A bulk composition in the form of a powder was prepared to produce bags of powdered composition. Each bag contained over 30 servings of the composition, in which each ingredient is present in the amounts shown in Table 3 per 3.765 gram serving. The powdered composition was prepared as described: First, each component listed in Table 3 above was weighed out and added to a stainless steel mixing bowl, followed by whisking each component with a stainless steel whisk until smooth. The whisked component was placed into an electric grinder (Krups) and blended finely, ground until no visible aggregates and clumps remain. Each ground component was deposited in a Vitamix blender model VM0102D (Vitamix, Cleveland, Ohio). Once all of the ground components had been placed in the Vitamix blender, the combined components were blended to form a powder having substantially uniform color and consistency. The blended powdered composition was placed in a 10 gallon mixing container and tumbled to further homogenize the powdered composition. The powdered composition was then deposited into bags and heat sealed.

Example 3

Nitric Oxide activation was tested of a composition containing of L-arginine, luo han guo extract containing mogrosides, resveratrol, and curcumin.

Six fasting (i.e., 10 hours of abstention from eating) human test subjects were tested for their baseline levels of nitric oxide upon awakening with a Berkeley Test, Nitric Oxide Saliva

Test Strips (Berkely Life, Chicago, Ill.), which is a non-invasive saliva test. The strips are based on the well-established Greiss reagent reaction that measures the conversion of nitrates to nitrite. To perform this test, each subject A) held a test strip on the tongue for five seconds before B) folding the test strip over to allow the saliva to cover the measurement portion of the strip. The average baseline level of all the test subjects registered was found to be considered “Low”, which was quantified at a level of 100 micromoles per liter (μM) of nitrate.

Next, each test subject ingested 1 dose of a mixture containing 1 g of L-arginine, 200 mg of luo han guo extract containing mogrosides, 20 mg of resveratrol, and 100 mg of curcumin, all mixed lightly in tap water. Subjects then waited 45 minutes and performed light standard office work computer tasks. After 45 minutes, the test subjects completed another test using the Berkeley Test, Nitric Oxide Saliva Test Strips using the same steps A) and B) described above. The average post-dose level of all the test subjects registered was found to be considered “Threshold”, which was quantified at a level of 220 μM. This shows that bio-conversion of the ingested mixture did occur after only 45 minutes, which exhibits activation and stimulation of downstream live nitric oxide within the human body.

While the specification has described in detail certain exemplary embodiments, it will be appreciated that those skilled in the art, upon attaining an understanding of the foregoing, may readily conceive of alterations to, variations of, and equivalents to these embodiments. Furthermore, all publications and patents referenced herein are incorporated by reference in their entirety to the same extent as if each individual publication or patent was specifically and individually indicated to be incorporated by reference. Various exemplary embodiments have been described. These and other embodiments are within the scope of the following claims.

What is claimed is:

1. A composition comprising:
a) L-arginine;
b) resveratrol;
c) curcumin; and
d) luo han guo extract comprising mogrosides.

2. The composition of claim 1, further comprising water, and wherein each of a) through d) is dissolved in the water.

3. The composition of claim 2, wherein the water is alkaline water and the composition has a pH of 7.1 or greater.

4. The composition of claim 1, in which each of a) though d) is blended together and the composition is in the form of a powder.

5. The composition of claim 1, further comprising beta-hydroxybutyrate (BHB).

6. The composition of claim 1, further comprising at least one amino acid selected from the group consisting of L-leucine, L-lysine, L-valine, L-isoleucine, L-threonine, L-phenylalanine, L-methionine, L-histidine, and L-tryptophan.

7. The composition of claim 6, comprising each of L-leucine, L-lysine, L-valine, L-isoleucine, L-threonine, L-phenylalanine, L-methionine, L-histidine, and L-tryptophan.

8. The composition of claim 7, wherein the amino acids are present in a ratio of 51 parts by weight of L-leucine: 22 parts by weight of L-lysine: 22 parts by weight of L-isoleucine:
12 parts by weight L-threonine: 7.5 parts by weight of L-phenylalanine: 3.5 parts by weight of L-methionine: 2.2 parts by weight L-histidine: 1 part by weight L-tryptophan.

9. The composition of claim 7, wherein each amino acid is present in an amount of 100 mg or greater per serving.

10. The composition of claim 1, further comprising at least one of theanine, glycine, or arginine.

11. The composition of claim 10, comprising each of theanine, glycine, and arginine.

12. The composition of claim 1, further comprising at least one of magnesium, zinc, sodium, or potassium.

13. The composition of claim 1, wherein the L-arginine is present in an amount of 400 mg or greater per serving, the resveratrol is present in an amount of 2 mg or greater per serving, the curcumin is present in an amount of 100 mg or greater per serving, and the luo han guo extract is present in an amount of 99 mg or greater per serving.

14. The composition of claim 1, wherein the L-arginine, curcumin, luo han guo extract, and resveratrol are present in a ratio of 8 parts by weight L-arginine: 2 parts by weight curcumin: 2 parts by weight luo han guo extract: 1 part by weight resveratrol.

15. A method of making a composition, the method comprising:
a) heating a first composition comprising water to a temperature of 165 to 180 degrees Fahrenheit;
b) mixing curcumin with the first composition having a temperature of 165 to 180 degrees Fahrenheit to form a second composition;
c) mixing luo han guo extract comprising mogrosides into the second composition having a temperature of 145 to 150 degrees Fahrenheit to form a third composition;
d) mixing resveratrol and L-arginine into the third composition having a temperature of 120 to 130 degrees Fahrenheit to form a fourth composition; and
e) mixing the fourth composition to form a solution.

16. A method of improving circulation in a subject, the method comprising:
a) administering to the subject a composition comprising:
1) L-arginine;
2) resveratrol;
3) curcumin; and
4) luo han guo extract comprising mogrosides.

17. The method of claim 16, wherein the composition is administered to the subject at least once daily.

18. The method of claim 16, wherein the improving circulation comprises increasing 5′ adenosine monophosphate-activated protein kinase (AMPK) activation.

19. The method of claim 16, wherein the improving circulation comprises improving blood flow.

20. The method of claim 16, wherein the improving circulation comprises increasing nitric oxide (NO) stimulation, NO synthesis, or both.
